(19) United States
(12) Patent Application Publication
(10) Pub. No.: US 2018/0055747 A1
(43) Pub. Date: Mar. 1, 2018

(54) A Tanning Composition

(71) Applicant: PZ Cussons Beauty LLP, London (GB)

(72) Inventor: Paul Done EVANS, London (GB)

(21) Appl. No.: 15/556,697

(22) PCT Filed: Mar. 11, 2016
(86) PCT No.: PCT/GB2016/050676

§ 371 Date: Sep. 8, 2017

(30) Foreign Application Priority Data
Mar. 11, 2015 (GB) 1504129.6

(51) Int. Cl. A61K 8/35; A61K 8/92; A61K 8/81; A61K 8/46; A61Q 19/04

(52) U.S. Cl. CPC A61K 8/35; A61K 8/922; A61K 8/8152; A61K 8/466; A61K 2800/5422; A61K 2800/52; A61K 2800/5424; A61K 2800/5428; A61Q 19/04

(57) ABSTRACT

A composition comprising 5 to 30% w/w skin tanning agent, 5 to 30% w/w oil, 1 to % w/w emulsifying and stabilizing agent, 7 to 15% w/w anionic surfactant, and water.

### FIELD OF THE INVENTION

The present invention is directed to a composition that produces a tanning or darkening effect on the skin. The composition is particularly suitable for use in the shower (in-shower). The composition includes and effectively delivers a tanning agent, such as dihydroxyacetone (DHA), to the skin.

### BACKGROUND OF THE INVENTION

A tanned or darkened skin is desirable in many global markets and is considered fashionable or giving a healthy appearance. Natural tanning through exposure to Ultra Violet rays (UV) either in sunbathing or by the use of artificial Ultra Violet lamps is widely practised, but there is a growing recognition of the longer term impacts of UV exposure such as premature skin-ageing and skin cancer. Therefore the market for products that darken the skin without exposure to UV is important and products in various formats have been developed and have been on the market for many years.

Self-tanning products containing tanning agents, such as dihydroxyacetone (DHA), have been marketed for many years in various topical application formats such as lotions, mousses, creams and sprays. Such products are left on the skin to dry allowing the tanned appearance to develop. The skin colouring effect of DHA was discovered by chance in a childrens' hospital where it was being used to treat diabetics who were more tolerant to DHA than glucose. Since that time the mode of action and consumer safety of DHA has been widely studied and there is a sizeable global market for self-tanning cosmetic products containing DHA which are seen as a safer tanning alternative than UV exposure through natural sunlight and sunbeds. The skin colouration comes from a reaction of DHA with the amino acids and amino groups present in the surface layers of the stratum corneum to produce brownish pigments (melanoids) through the well-known Maillard type reaction. The melanoids produce a cosmetically appealing skin colour similar to that produced by UV exposure.

There are existing marketed products designed to deliver different levels of tan and speed of reaction to meet consumer demands. These range from ‘gradual’ or ‘everyday’ products designed to be used daily and deliver a low level tan to ‘dark’ products which deliver a much higher level of skin colouration from a single application.

However, products containing DHA often produce an uneven or streaky tan appearance. The application technique and preparation of the skin, using products such as exfoliators and moisturisers, are therefore important to achieve the most cosmetically appealing appearance. As an aid to producing an even tan colouration, most self-tan products contain cosmetic pigments which act as a ‘guide’ colour and are visible on the skin. These are a visual aid to help ensure even coverage all over the desired tanning area and are showered off typically about eight hours after application. The user must allow the product to dry before dressing or getting into bed to avoid staining of clothes or bedsheets. The tanning regime can therefore be time consuming and require preparation of the skin to produce a natural looking tan appearance.

In addition products containing DHA produce a characteristic unpleasant odour which is produced by the Maillard reaction. This odour cannot be removed, but product developers' work to mask the odour produced using specially designed fragrance ingredients. This unpleasant odour can put off some consumers from using these products.

Some consumers find the time required to apply self-tan products (application time) and the development time an undesirable aspect. The amount of development time can vary from 30 minutes to up to eight hours.

### SUMMARY OF THE INVENTION

In a first aspect, the present invention provides a composition comprising:
5 to 30% w/w skin tanning agent;
5 to 30% w/w oil;
1 to 5% w/w emulsifying and stabilizing agent;
7 to 15% w/w anionic surfactant; and water.

The invention provides a cosmetically acceptable sunless or self-tanning product composition. The composition is particularly applicable for use in the shower.

The composition is suitable for use in the shower as part of a daily cleansing regime and is easy to spread, apply and rinse.

The tanning agent contained in the composition causes a chemical reaction in the surface layers of the skin of the user to produce the brown colouration.

A suitable tanning agent is dihydroxyacetone (DHA).

The tanning agent may be present in the composition in an amount of 8 to 27% w/w, preferably 10 to 25% w/w, more preferably 15 to 23% w/w, for example 18 to 22% w/w. The tanning agent may be present in an amount of up to 22.5%, for example 10 to 22.5%, 15 to 22.5% or 18 to 22.5%.

This is deemed a high level of DHA as compared to existing leave-on tanning compositions such as lotions, creams and sprays. High levels of dihydroxyacetone increase the instability of the composition. The composition of the present invention is stable despite high levels of DHA due to the surfactant and emulsifying and stabilising agent.

The oil in the composition acts as a skin-conditioning agent; it is a moisturiser and contributes to the texture and cosmetic appeal of the composition.

The oil may be a single oil or may comprise two or more oils. The oil may be present in an amount of 7 to 25% w/w, preferably 8 to 20% w/w, more preferably 9 to 18% w/w, for example 10 to 15% w/w.

This is considered a high amount of oil compared to existing surfactant based shower products, most shower products do not contain any oil. The high level of oil contributes to the performance of the product and produces a very good skin feel after use.

Any suitable oil could be used in the composition. The type of oil is only limited by the need to produce a cosmetically pleasing and stable composition.

The oil could comprise a natural fruit based oil, mineral oil, silicone oil or combination thereof which gives a stable and cosmetically pleasing composition.

In one embodiment the oil is a natural fruit oil. Examples of suitable natural fruit oils include sweet almond oil, sunflower oil, apricot kernel oil, avocado oil, babassu oil, coconut oil, brazil nut oil, olive oil, sesame oil, hemp oil, marula oil, jojoba oil, peach kernel oil, papaya seed oil, grape seed oil and combinations thereof.

In one embodiment the oil is sweet almond oil (Prunus Amygdalus Dulcis).

In one embodiment the oil is a silicone oil. Examples of suitable silicone oils include dimethicone, cyclopentasiloxane, cyclomethicon, amodimethicone, dimethiconol, phenyl trimethicone, siloxane, cyclohexasiloxane, peg-12 dimethicone and combinations thereof.

The anionic surfactant is used in the composition to stabilise the composition and to provide suitable foaming properties, including lathering to aid rinsing.

The anionic surfactant may be present in an amount of 7 to 15% w/w, preferably 8 to 13% w/w, more preferably 9 to 11% w/w.

The anionic surfactant may comprise a carboxylate, a sulphate, a sulfosuccinate or a combination thereof.

In one embodiment the anionic surfactant is a carboxylate, for example sodium lauryl glucose carboxylate.

In one embodiment the anionic surfactant is a sulphate. Examples of suitable sulphates include sodium laureth sulphate, sodium myreth sulphate and ammonium lauryl sulphate.

In another embodiment the anionic surfactant is a sulphosuccinate. Examples of suitable sulphosuccinates include diethylhexyl sodium sulfosuccinate, disodium laureth sulfosuccinate and disodium lauryl sulfosuccinate.

In one embodiment the anionic surfactant is diethylhexyl sodium sulfosuccinate.

The emulsifying and stabilising agent is any suitable compound that emulsifies the oil phase and thickens and stabilises the composition, preventing the oil phase from coalescing. The emulsifying and stabilising agent creates a gel-like structure and texture for the composition. The emulsifying and stabilising agent is essential to the texture of the composition and the overall composition stability. The emulsifying and stabilising agent also assists with the stabilization of dihydroxyacetone when this tanning agent is used in the composition.

The emulsifying and stabilising agent may be present in an amount of 1.5 to 4% w/w, for example 2 to 4% w/w.

The emulsifying and stabilising agent may comprise a suitable wax, polymer or combination thereof.

The emulsifying and stabilising agent may comprise a crosslinked polymer or a copolymer, the crosslinked polymer or copolymer being comprised of monomers of at least one of acrylic acid, sodium acrylate, methacrylic acid or alkyl acrylate

In one embodiment the emulsifying and stabilising agent is a crosslinked polymer, particularly a crosslinked polymer comprised of monomers of at least one of acrylic acid, sodium acrylate, methacrylic acid and alkyl acrylate. Examples of suitable crosslinked polymers include acrylates/C₁₀₋₃₀ alkyl acrylate crosspolymer, acrylates crosspolymer and carbomer

In another embodiment the emulsifying and stabilising agent is a copolymer, particularly a copolymer comprised of at least one of acrylic acid, sodium acrylate, methacrylic acid and alkyl acrylate. Examples of suitable copolymers include acrylates copolymer, sodium acrylate/sodium acryloyldimethyl taurate copolymer (which is a copolymer of sodiumacrylate and sodium acryloyldimethyl taurate monomers) and acrylates/steareth-20 methacrylate copolymer.

In one embodiment the emulsifying and stabilising agent is sodium acrylate/sodium acryloyldimethyl taurate copolymer.

The emulsifying agent may comprise a wax. Suitable waxes may include mixtures of fatty alcohols, esterification of materials or polyethylene glycol ethers particularly cetearyl alcohol, glyceryl stearate and ceteareth-20 respectively.

As previously explained, in current self-tanning products the amount of development time can vary from 30 minutes to up to eight hours and some consumers find this time an undesirable aspect.

The present invention however provides a composition with improved development times. The composition of the invention can deliver an amount of tanning agent, such as DHA, sufficient to produce at least a light tanned appearance when allowing the composition to remain in contact with the skin for up to 6 minutes, preferably for 1 to 6 minutes, more preferably 2 to 5 minutes and more preferably 2 to 4 minutes, for example 3 minutes.

In one embodiment the composition does not thicken on contact with water. Typically the composition will also contain other ingredients necessary to produce a cosmetic product.

The composition may comprise one or more further surfactants.

The composition may comprise an amphoteric surfactant. The amphoteric surfactant may be present in an amount of 3 to 10% w/w, preferably 4 to 8% w/w, more preferably 5 to 7% w/w. The amphoteric surfactant may be present in an amount of 4 to 5% w/w or 4.1 to 4.3% w/w, for example 4.16% w/w. The amphoteric surfactant may be present in an amount of 5 to 6% w/w or 5.1 to 5.3% w/w, for example 5.16% w/w.

Suitable amphoteric surfactants include capryl/capram idopropyl betaine, cocamidopropyl betaine and sodium lauriminodipropionate. In one embodiment the amphoteric surfactant is cocamidopropyl betaine. The amphoteric surfactant acts as a foam booster and foam stabiliser, a composition stabiliser and assists in the provision of lathering to aid rinsing.

The non-ionic surfactant may be present in an amount of 1 to 9% w/w, preferably 1.5 to 7% w/w, more preferably 2.5 to 4% w/w.

Suitable non-ionic surfactants include lauryl glucoside, decyl glucoside, caprylyl/capryl glucoside and coco-glucoside. In one embodiment the non-ionic surfactant is decyl glucoside. The non-ionic surfactant acts as a composition stabiliser and provides lathering to aid rinsing.

In one embodiment the composition further comprises at least one amphoteric surfactant and/or at least one non-ionic surfactant. In one embodiment the composition further comprises at least one amphoteric surfactant and at least one non-ionic surfactant. The amphoteric surfactant may be cocamidopropyl betaine and the non-ionic surfactant may be decyl glucoside.

The composition may further comprise an emollient or penetration enhancer. Said emollient or penetration enhancer may be present in the composition in an amount of up to 2% w/w, preferably up to 1.5% w/w, more preferably up to 1% w/w. Said emollient or penetration enhancer may be present in the composition in an amount from 0.1% to 2% w/w, preferably 0.5 to 1.5% w/w, more preferably 0.7 to 1.2% w/w.

A suitable emollient or penetration enhancer may be a cosmetic grade glycol material, such as pentylene glycol or butylene glycol or a suitable natural oil. Preferably, the emollient or penetration enhancer is a jojoba ester, such as hydrolysed jojoba esters. The use of an emollient or penetration enhancer helps enhance the tanning effect. It is believed that the emollient or penetration enhancer, particularly jojoba esters, enhances the release of the tanning agent such as DHA from the composition. It is believed that the emollient or penetration enhancer enhances deposition of tanning agents such as DHA on the skin aiding penetration of and adherence to the skin. The presence of suitable emollients or penetration enhancers assists in enabling the tanning reaction to occur within a wait time of up to 6 minutes, for example from 1 to 6 minutes, from 2 to 5 minutes, from 2 to 4 minutes, for example 3 minutes.

The composition may also include a cellulose derivative, such as hydroxyethylcellulose, or a gum, such as xanthan gum or gelatin, in an amount of 0.01% to 1.0%. These materials thicken the water phase of the composition further contributing to the overall stability of the composition.

The composition may further include ethoxydiglycol in an amount of from 0.5 to 10% w/w, preferably 0.5 to 8% w/w, more preferably 0.5 to 6% w/w, more preferably 1 to 4% w/w, for example 1 to 2% w/w. The ethoxydiglycol acts as a solvent for dihydroxyacetone.

Supplementary tanning agents such as erythrulose or melanin may also be incorporated into the composition, for example in an amount of from 1 to 3% w/w, to improve the quality or natural appearance of the developed skin colour. Supplementary tanning agents such as erythrulose or melanin may also be incorporated into the composition in an amount of from 0.05 to 3% w/w. Preferably, supplementary tanning agents are present in an amount of 0.05 to 2.5% w/w, preferably 0.05 to 2% w/w or 0.1 to 2% w/w. Where the supplementary tanning agent is erythrulose it may be present in an amount of 1.5 to 2.5% w/w, preferably 2% w/w. Where the supplementary tanning agent is melanin it may be present in an amount of from 0.05 to 0.5% w/w, preferably from 0.05% w/w to 0.25% w/w, for example 0.1% w/w.

The composition may include one or more suitable fragrances. The fragrance may be present in an amount of 0.1 to 3% w/w.

The composition may further include one or more of polysorbates, polymers of ethylene oxide or polymers of propylene oxide (such as PPG-1 Trideceth-6, PPG-5-Ceteth-20, PEG-40 Hydrogenated Castor Oil) and PEG-18 glyceryl oleate/cocoate. Each of these materials may be present in an amount of up to 2% w/w. In one embodiment the composition includes PEG-18 glyceryl oleate/cocoate. In this embodiment the PEG-18 glyceryl oleate/cocoate is present on an amount of from 0.5 to 2% w/w. These materials act as an emollient skin conditioning agent and a thickener for foaming products. When used with a fragrance they help to solubilise the fragrance and give a thicker texture to the composition.

The composition may further include one or more compounds that when used with a fragrance help to increase viscosity of the composition. Suitable compounds include coco-glucoside and glycerol oleate. The compounds that when used with a fragrance help to increase viscosity of the composition may be present in an amount of 0.2 to 3% w/w, for example 0.5 to 2% w/w. Each of coco-glucoside and glycerol oleate may be present in an amount of 0.1 to 1.5% w/w, for example 0.25 to 1% w/w.

The composition may include an antioxidant. The antioxidant may be present in an amount of from 0.01 to 0.1% w/w, for example 0.05 to 0.1% w/w. Suitable antioxidants may include pentaerythrityl tetra-DI-t-butyl hydroxyhydrocinnamate. The use of antioxidants helps prevent discolouration of the composition.

The composition may include other additives to produce desired or appealing characteristics for a cosmetic product. These may be humectants, skin conditioning agents or emollients and may be selected from a wide range of cosmetic ingredients.

The humectant may be present in an amount of up to 10% w/w. Suitable humectants include glycerine, sorbitol, propylene glycol, dipropylene glycol or butylene glycol. Humectants add moisture to the skin and work in addition to the oil to impart a moisturised feel to the skin after using the composition.

Suitable skin conditioning agents or emollients may be present in an amount of 0.1% to 10%. Suitable skin conditioning agents or emollients may include decylene glycol, caprylyl glycol, mineral oils or isohexadecane.

The composition may include an antimicrobial to protect the composition against the risk of microbial contamination. Typically antimicrobials are included in the composition at up to 1.0%. A chemist skilled in the art of cosmetic product development and preservation would select an antimicrobial system appropriate for the composition and the microbial challenge. Suitable antimicrobials may include one or more of paraben esters (methyl, ethyl, propyl, isopropyl, butyl, isobutyl) at 0.1-0.3% in combination, DMDM hydantoin at concentration of 0.15-0.4%, imidazolidinyl urea at typical use concentration 0.2-0.5%, benzoic acid, sodium benzoate at 0.2-0.4 typical use, potassium sorbate at 0.3-0.5% and benzyl alcohol at 0.4-1.0

The composition may include a colouring agent, for example a brown colouring agent such as caramel at 0.1% to 3.0%.

The composition may include sodium chloride in an amount of 0.1% to 1.0%. The sodium chloride acts to assist controlling viscosity of the composition.

In one embodiment the composition of the present invention comprises
5 to 30% w/w skin tanning agent;
5 to 30% w/w oil;
1 to 5% w/w emulsifying and stabilizing agent;
7 to 15% w/w anionic surfactant;
up to 2% w/w emollient or penetration enhancer; and
water.

In one embodiment the composition of the present invention comprises
5 to 30% w/w skin tanning agent;
5 to 30% w/w oil;
1 to 5% w/w emulsifying and stabilizing agent;
7 to 15% w/w anionic surfactant;
3 to 10% w/w amphoteric surfactant; and
water.

In one embodiment the composition of the present invention comprises
5 to 30% w/w skin tanning agent;
5 to 30% w/w oil;
1 to 5% w/w emulsifying and stabilizing agent;
7 to 15% w/w anionic surfactant;
3 to 10% w/w amphoteric surfactant;
up to 2% w/w emollient or penetration enhancer; and
water.

In one embodiment the composition of the present invention comprises
5 to 30% w/w skin tanning agent;
5 to 30% w/w oil;
1 to 5% w/w emulsifying and stabilizing agent;
7 to 15% w/w anionic surfactant;
3 to 10% w/w amphoteric surfactant;
up to 2% w/w emollient or penetration enhancer;
0.05 to 3% w/w supplementary tanning agent; and
water.

Use of showers is now the most common daily cleansing regime in many markets. In the UK for example, about 60% of users of cleansing products shower every day. There is also common acceptance that consumers use a range of products other than the primary cleansing product during showering and use the showering process as part of their daily skincare regime. Other products commonly used in the shower include exfoliators, shampoos, conditioners, shaving products, facial cleansers.

The functional benefits that can be delivered from a showering product are limited when compared to a leave-on cosmetic product. It is known that the showering process, rinsing and towelling dry action remove most of the ingredients from the skin. In safety assessments of showering products a very low retention factor of 0.1 is used to assess the safety profile of ingredients. Products which have been designed to deliver functional benefits in the shower have often had poor cosmetic or in-use attributes such as greasy or oily skin feel and difficult to rinse off.

It is of increasing interest to develop a composition suitable to deliver dihydroxyacetone to the skin with a faster application and development time, and particularly, where the composition suitable for use in the shower and is easy to spread, apply and rinse. This invention, addresses this aim.

The composition of the present invention is an oil-in-water composition. The composition contains a high amount of oil and tanning agent, for example dihydroxyacetone. The composition is in one embodiment an oil-in-water gel-emulsion. The composition has been specifically designed to deliver an amount of tanning agent, such as DHA, to the skin sufficient to produce the desired colouring effect on the skin within the showering regime.

The benefit of an oil-in-water composition is that the tanning agent, such as DHA, is in the external facing part of the composition and is therefore in contact with the skin rather than being held within the composition unable to easily contact the skin as would be the case in a water-in-oil composition. Increasing the contact area increases the reaction time with the skin which is a key attribute of the contribution for its performance in tanning the skin. The product must be able to react with the skin quickly which is an aspect able to happen in an oil-in-water composition.

Further benefits are for the functionality of the product being an oil-in-water composition as it is working in conjunction with the water left on the skin from showering. The external phase contains surfactants and water soluble ingredients allow the product to be applied, spread, remain in contact for development time and aid to easy rinsing. A water-in-oil composition would resist the water on the skin, creating an unusable product.

With regular use in the shower, the composition produces an even, natural looking tan colouration of the skin. The product has cosmetically acceptable properties and is pleasant to use. The product spreads evenly and easily across the skin, it also contains foaming agents to enable easy rinsing off the skin.

In a second aspect, the present invention provides a method of delivering a tanning agent to the skin of a user comprising the steps of:
applying a composition of the first aspect of the invention to the skin; and
allowing the composition to remain in contact with the skin for up to 6 minutes.

In one embodiment the method further comprises subjecting skin to exposure to warm water before applying a composition of the first aspect of the invention to the skin.

In one embodiment the method can comprise rinsing the composition off the skin after allowing the composition to remain in contact with the skin for up to 6 minutes.

The step of subjecting the skin to exposure to warm water may comprise showering. Showering can be conducted in the normal way with any cleansing product.

The composition is preferably applied evenly over the skin. The composition is preferably applied to skin which is wet but no longer exposed to water.

The composition is preferably allowed to remain in contact with the skin for 1 to 6 minutes, preferably 2 to 5 minutes, more preferably 2 to 4 minutes, for example 3 minutes. In one embodiment the user remains in the area of the shower with the water switched off during this time.

The composition may be rinsed off the skin by showering. The composition may be lathered and rinsed off.

The composition of the first aspect of the invention delivers an amount of tanning agent, such as DHA, sufficient to produce a light tanned appearance when used as directed in the shower.

The composition is cosmetically acceptable, pleasant to use in the shower and easily rinses off.

In one embodiment the composition is stable and meets the normal demands of the cosmetic market. In this embodiment the product should meets the intended physical, chemical and microbiological quality standards as well as functionality and aesthetics when stored under appropriate conditions.

The applicant believes that the application of the tanning composition of the present invention as part of the showering process is beneficial as the colouration performance achieved from the composition is greater than expected.

A normal shower cleansing product, either liquid or bar format, is used to cleanse the skin removing excess oils, dirt and dead skin cells leaving the skin in a consistent, even state prior to application of the self-tanning composition. This produces even spread of the composition and a more desirable even and streak free skin colouration.

In addition, during the showering process, which uses typical water temperatures of between 35° C. and 45° C., the skin becomes receptive to the action of a tanning agent, such as DHA. In addition, at these temperatures of use the tanning agent, such as DHA is more effective.

In addition other benefits of an in-shower product are:

Leave-on self-tanning products often contain colours which are used to highlight which parts of the body have been covered or not. These colours can stain clothing and bedclothes if not allowed to dry properly. By applying the product in the shower, then lathering/rinsing off the product fully, this problem does not occur.

The DHA reaction with the skin produces a slightly unpleasant odour (to most people) which is characteristic of these products. By applying the product in the shower then rinsing off excess product, the noticeability of this odour is very much less than expected.

Embodiments of the invention are described by means of the example compositions below. The example compositions are purely illustrative and are not intended to limit the scope of protection in any way.

### Examples of Possible Compositions of the Invention Include:

### Example Composition 1

20% w/w dihydroxyacetone;
20% w/w sweet almond oil;
3% w/w sodium acrylate/sodium acryloyldimethyl taurate copolymer
10% w/w diethylhexyl sodium sulfosuccinate; and
balance w/w water

### Example Composition 2

20% w/w dihydroxyacetone;
20% w/w sweet almond oil;
2-3% w/w sodium acrylate/sodium acryloyldimethyl taurate copolymer;
9-11% diethylhexyl sodium sulfosuccinate;
4.5-5% w/w cocamidopropyl betain;
2.5-3% w/w decyl glucoside;
0.1-0.3% w/w hydroxyethylcellulose; and
balance water up to 100%.

Embodiments of the present invention will now be described, purely by way of example, with reference to the accompanying drawings, in which:

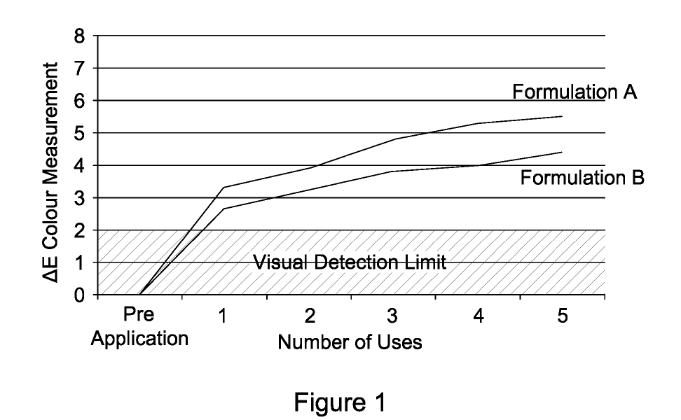

FIG. 1—illustrates the results of a study in which example compositions of the invention were tested in a panel of 20. The graph shows the colour measurement AE value per number of uses of the compositions.

### EXAMPLE

Compositions according to the invention were tested in a controlled panellist study using the following application method:

All panellists showered in their normal manner using a standard gel. A panel of 20 were then randomly split into two equal groups. The allocation of test materials was randomised to eliminate position and order bias by a computer system which produces a Randomised Product Application Schedule for each subject on the study.

Test compositions A and B were prepared and tested.

| | Test composition A | Test composition B |
|---|---|---|
| Dihydroxyacetone (DHA) | 21-23% | 18-22% |
| Almond Oil | 14-16% | 14-16% |
| DiethylHexylSulfosuccinate | 9-11% | 9-11% |
| CocamidoPropylBetaine | 4-5% | 4-5% |
| Melanin | 0-0.3% | — |
| Hydrolyzed Jojoba Esters | 0.1-1.0% | — |
| Decyl Glucoside | 2.5-3.0% | 2.5-3.0% |

Half the panel then applied the test composition A to one leg and a standard shower gel (control) to the other leg—both products were then lathered and showered (rinsed) off in the normal manner and towel dried.

The other half of the panel applied the test composition B to one leg and a standard shower gel (control) to the other leg—both products were then lathered and showered (rinsed) off in the normal manner and towel dried.

Test compositions A and B were applied and left on skin for 3 minutes. The test and control compositions were applied in a circular motion and massaged into wet skin, creating an even coverage. After application, panellists washed their hands concentrating on their palms, nails and in-between fingers to avoid staining. After 3 minutes, panellists stepped back into the shower to lather up and rinse off the test and control compositions.

The study was carried out over 7 days with the test product applied on 5 occasions.

Skin colour was assessed visually by trained assessors and also using a Cortex Colormeter which uses the L*a*b* numerical system to quantify colour of the skin. This is a system commonly used for colour measurement, and gives a measure of tone, intensity and luminosity. Differences in colour produced by the composition were measured tracking changes in a* and b* which quantify tone/intensity and L which quantifies luminosity.

To demonstrate the overall impact on colour the combination parameter, ΔE was used:

ΔE=√Δa*2+Δb*2+ΔL2

ΔE values are commonly used to quantify colour differences in many industries, most people can typically detect a difference in ΔE of 2 units.

As can be seen in FIG. 1, most panellists showed a difference in ΔE>2 after the first application. This value is commonly accepted as the visual detection limit.

The above compositions provide a cosmetically acceptable sunless or self-tanning product. The compositions are particularly applicable for use in the shower. The compositions deliver the DHA to the skin and allow it to produce the desired colouring effect on the skin within the showering regime.

1. A composition comprising:
5 to 30% w/w skin tanning agent;
5 to 30% w/w oil;
1 to 5% w/w emulsifying and stabilizing agent;
7 to 15% w/w anionic surfactant; and
water.

2. The composition of claim 1, wherein the skin tanning agent is dihydroxyacetone.

3. The composition of claim 1 or claim 2, wherein the skin tanning agent is present in the composition in an amount of 15 to 23% w/w.

4. The composition of any one of claims 1 to 3, wherein the oil is present in an amount of 8 to 20% w/w.

5. The composition of any one of claims 1 to 4, wherein the oil comprises a natural fruit based oil, mineral oil, silicone oil or combination thereof.

6. The composition of claim 5, wherein the natural fruit based oil is selected from sweet almond oil, sunflower oil, apricot kernel oil, avocado oil, babassu oil, coconut oil, brazil nut oil, olive oil, sesame oil, hemp oil, marula oil, jojoba oil, peach kernel oil, papaya seed oil, grape seed oil and combinations thereof.

7. The composition of claim 5, wherein the silicone oils is selected from dimethicone, cyclopentasiloxane, cyclomethicon, amodimethicone, dimethiconol, phenyl trimethicone, siloxane, cyclohexasiloxane, peg-12 dimethicone and combinations thereof.

8. The composition of any one of claims 1 to 7, wherein the emulsifying and stabilising agent is present in an amount of 2 to 4% w/w.

9. The composition of any one of claims 1 to 8, wherein the emulsifying and stabilising agent comprises a wax, polymer or combination thereof.

10. The composition of claim 9, wherein the emulsifying and stabilising agent comprises a crosslinked polymer or a copolymer, the crosslinked polymer or copolymer being comprised of monomers of at least one of acrylic acid, sodium acrylate, methacrylic acid or alkyl acrylate.

11. The composition of claim 9, wherein the emulsifying and stabilising agent is selected from acrylates/C₁₀₋₃₀ alkyl acrylate crosspolymer, acrylates crosspolymer, carbomer, acrylates copolymer, sodium acrylate/sodium acryloyldimethyl taurate copolymer, acrylates/steareth-20 methacrylate copolymer, mixtures of fatty alcohols, esterification of materials and polyethylene glycol ethers.

12. The composition of any one of claims 1 to 11, wherein the anionic surfactant is present in an amount of 8 to 13% w/w.

13. The composition of any one of claims 1 to 12, wherein the anionic surfactant comprises a carboxylate, a sulphate, a sulfosuccinate or a combination thereof.

14. The composition of claim 13, wherein the anionic surfactant is selected from sodium lauryl glucose carboxylate, sodium laureth sulphate, sodium myreth sulphate, ammonium lauryl sulphate, diethylhexyl sodium sulfosuccinate, disodium laureth sulfosuccinate and disodium lauryl sulfosuccinate.

15. The composition of any one of claims 1 to 14 further comprising an amphoteric surfactant.

16. The composition of claim 15, wherein the amphoteric surfactant is present in an amount of 3 to 10% w/w.

17. The composition of claim 15 or claim 16, wherein the amphoteric surfactant is selected from capryl/capram idopropyl betaine, cocamidopropyl betaine and sodium lauriminodipropionate.

18. The composition of any one of claims 1 to 17 further comprising a non-ionic surfactant.

19. The composition of claim 18, wherein the non-ionic surfactant is present in an amount of 1 to 9% w/w.

20. The composition of claim 18 or claim 19, wherein the non-ionic surfactant is selected from lauryl glucoside, decyl glucoside, caprylyl/capryl glucoside and coco-glucoside.

21. The composition of any one of claims 1 to 20 further comprising an emollient or penetration enhancer.

22. The composition of claim 21, wherein the emollient or penetration enhancer is present in the composition in an amount of up to 2% w/w.

23. The composition of claim 21 or claim 22, wherein the emollient or penetration enhancer is a jojoba ester.

24. The composition of any one of claims 1 to 23 further comprising from 0.05 to 3% w/w of a supplementary tanning agent.

25. The composition of claim 24, wherein the supplementary tanning agent is erythrulose or melanin.

26. The composition as defined in any one claims 1 to 25 for use as or in a sunless or self-tanning product.

27. The composition as defined in any one claims 1 to 25 for use in the shower or as a shower product.

28. A sunless or self-tanning product comprising the composition as defined in any one of claims 1 to 25.

29. A shower product comprising the composition as defined in any one of claims 1 to 25.

30. A method of delivering a tanning agent to the skin of a user comprising the steps of:
i) applying a composition as defined in any one of claims 1 to 25 to the skin, and
ii) allowing the composition to remain in contact with the skin for up to 6 minutes.

31. The method of claim 30 wherein the composition is allowed to remain in contact with the skin for 1 to 6 minutes.

32. The method as defined in claim 30 or 31 further comprising subjecting skin to exposure to warm water, for example by showering, before applying the composition.

33. A sunless or self-tanning product comprising a skin tanning agent, wherein the product provides a difference in skin colour of ΔE>2 measured by a Cortex Colormetera when the product is applied to the skin and allowed remain in contact with the skin for up to 6 minutes, for example for 1 to 6 minutes.

34. A product as defined in claim 33 for use in the shower or as a shower product.

35. A method of delivering a tanning agent to the skin of a user comprising the steps of:
i) applying a composition as defined in claim 33 to the skin, and
ii) allowing the composition to remain in contact with the skin for up to 6 minutes, for example from 1 to 6 minutes.

36. The method as defined in claim 35 further comprising subjecting skin to exposure to warm water, for example by showering, before applying the composition.

37. A composition substantially as described herein.

38. A method of delivering a tanning agent to the skin substantially as described herein.
